(19) United States
(12) Patent Application Publication
(10) Pub. No.: US 2025/0161085 A1
(43) Pub. Date: May 22, 2025

(54) CO-AXIAL DELIVERY SYSTEM FOR SELF-EXPANDING STENTS HAVING DISTAL RETENTION STRUCTURE

(71) Applicant: BOSTON SCIENTIFIC SCIMED, INC., Maple Grove, MN (US)

(72) Inventors: Martyn G. Folan, Galway (IE); Louis McNern, Donegal (IE); Kevin McEvilly, Galway (IE)

(73) Assignee: BOSTON SCIENTIFIC SCIMED, INC., Maple Grove, MN (US)

(21) Appl. No.: 18/952,612

(22) Filed: Nov. 19, 2024

Related U.S. Application Data
(60) Provisional application No. 63/600,993, filed on Nov. 20, 2023.

(51) Int. Cl. A61F 2/95; A61F 2/82

(52) U.S. Cl. CPC A61F 2/95; A61F 2/82; A61F 2002/9505

(57) ABSTRACT

An assembly for use in endoscopic stent delivery, including a stent delivery device and an associated stent loaded therein. The stent delivery device includes an outer tubular member, an inner member slidably disposed within the lumen of the outer tubular member, and a distal tip disposed at the distal end of the inner member. A self-expanding stent is positioned within the outer tubular member in a compressed configuration, with one or more retaining loops extending from the body of the stent and engaged with a retaining structure, such as a notch, provided by the distal tip. Upon deployment of the stent from the stent delivery device, proximal withdrawal of the distal tip through the lumen of the stent disengages the one or more retaining loops from the retaining structure.

### CROSS-REFERENCE TO RELATED APPLICATIONS

The present application claims the benefit of U.S. Provisional Patent Application Ser. No. 63/600,993, filed Nov. 20, 2023, the disclosure of which is incorporated herein by reference.

### TECHNICAL FIELD

The disclosure pertains to medical devices and more particularly to stent delivery systems including a distal stent retention structure.

### BACKGROUND

A wide variety of medical devices have been developed for medical use including, for example, medical devices utilized to deliver stents. These medical devices may be used in a variety of locations and are manufactured and used according to any one of a variety of different methods. Of the known medical devices and methods, each has certain advantages and disadvantages. In most cases, stent delivery may be limited depending on accessibility of a delivery location. There is an ongoing need to provide alternative stent delivery devices as well as alternative methods for manufacturing and using the stent delivery devices.

### SUMMARY

This disclosure provides design, material, manufacturing method, and use alternatives for medical devices.

A first example is a stent delivery system. The system includes an outer tubular member having a proximal end, a distal end, and a lumen extending to the distal end; an inner member slidably disposed within the lumen of the outer tubular member; a distal tip disposed at a distal end of the inner member, the distal tip including a retaining structure; and a self-expanding stent having a proximal end, a distal end, and a lumen extending therethrough. The stent is expandable from a radially compressed configuration to a radially expanded configuration. The stent includes one or more retaining loops disposed at the distal end of the stent. In the radially compressed configuration, the self-expanding stent is disposed between an outer surface of the inner member and an inner surface of the outer tubular member. The one or more retaining loops of the stent is configured engage with the retaining structure of the distal tip to maintain position of the distal end of the stent relative to the distal tip.

Alternatively or additionally to any of the examples above, in another example, the retaining structure comprises a notch opening out to a distal end of the distal tip.

Alternatively or additionally to any of the examples above, in another example, the retaining structure comprises at least one notch opening out to a side surface of the distal tip proximal a distal end of the distal tip.

Alternatively or additionally to any of the examples above, in another example, the distal tip further comprises a longitudinal groove extending along a side surface of the distal tip, wherein the one or more retention loops is configured to rest within the groove.

Alternatively or additionally to any of the examples above, in another example, the one or more retaining loops of the self-expanding stent comprise at least one elongated loop.

Alternatively or additionally to any of the examples above, in another example, the one or more retaining loops of the self-expanding stent comprise at least one retaining suture.

Alternatively or additionally to any of the examples above, in another example the system includes a guidewire slidably disposed within a lumen of the inner member.

Alternatively or additionally to any of the examples above, in another example, the distal tip of the inner member further includes a guidewire lumen through which the guidewire can extend in a distal direction past the distal end of the distal tip.

Alternatively or additionally to any of the examples above, in another example, the notch is off-centered relative to a central longitudinal axis of the distal tip, such that when the guidewire moves in a proximal or distal direction, the guidewire does not come in contact with the one or more retaining loops disposed within the notch.

Alternatively or additionally to any of the examples above, in another example, the distal tip further comprises a curved beak configured to partially cover the notch.

Alternatively or additionally to any of the examples above, in another example, the curved beak of the distal tip comprises a guidewire lumen.

Another example is a stent delivery system. The system includes an outer tubular member having a proximal end, a distal end, and a lumen extending to the distal end; an inner member slidably disposed within the lumen of the outer tubular member; a distal tip disposed at a distal end of the inner member, the distal tip including a notch; and a self-expanding stent having a proximal end, a distal end, and a lumen extending therethrough. The stent is expandable from a radially compressed configuration to a radially expanded configuration. The stent includes one or more retaining loops disposed at the distal end of the stent. In the radially compressed configuration, the self-expanding stent is disposed between an outer surface of the inner member and an inner surface of the outer tubular member. The one or more retaining loops of the self-expanding stent are positionable in the notch of the distal tip. The outer tubular member is configured to be withdrawn proximally relative to the stent to expose the stent for radial expansion to the radially expanded configuration. The one or more retaining loops is disposed within the notch to hold the distal end of the stent in place axially relative to the distal tip when the outer tubular member is withdrawn proximally.

Alternatively or additionally to any of the examples above, in another example, the notch opens out in a distal direction.

Alternatively or additionally to any of the examples above, in another example, the one or more retaining loops of the self-expanding stent are releasable from the notch when the distal tip is withdrawn proximally through the lumen of the stent.

Alternatively or additionally to any of the examples above, in another example, the distal tip further comprises a curved beak configured to partially cover the notch.

Alternatively or additionally to any of the examples above, in another example, the curved beak of the distal tip comprises a guidewire lumen configured to receive a guidewire therethrough.

Another example is a method of deploying a stent. The method includes advancing a distal end of a stent delivery device to a target location within a body lumen. The stent delivery device includes an outer tubular member, an inner member slidably disposed within the outer tubular member, and a distal tip disposed at a distal end of the inner member. A stent is loaded within the outer tubular member proximal of the distal tip. The stent includes one or more retaining loops engaged with a retention feature of the distal tip of the stent delivery device. The method includes withdrawing the outer tubular member of the stent delivery device in a proximal direction relative to the stent. Withdrawal of the outer tubular member from the stent exposes an entire length of the stent to permit a proximal end region of the stent to radially expand to an expanded configuration while the one or more retaining loops remain engaged with the retention feature of the distal tip. Thereafter, the inner member is withdrawn in the proximal direction to release the one or more retaining loops of the stent from the retaining structure of the distal tip.

Alternatively or additionally to any of the examples above, in another example, the retaining structure comprises a notch opening out in a distal direction.

Alternatively or additionally to any of the examples above, in another example, upon withdrawing the inner member, the self-expanding stent is held frictionally in place within the body, such that the one or more retaining loops are released from the notch.

Alternatively or additionally to any of the examples above, in another example, upon releasing the one or more retaining loops from the retaining structure, a distal end of the stent is allowed to radially expand against the body lumen.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a perspective view of an embodiment of a stent delivery system.

FIGS. 2A-2C illustrate a stent foreshortening when being deployed from a conventional stent delivery catheter.

FIG. 3A illustrates example strictures near an organ.

FIG. 3B illustrates stents positioned across the strictures shown in FIG. 3A.

FIGS. 4A-4E illustrate aspects of deploying a stent with a modified stent delivery device to prevent foreshortening.

FIG. 5A illustrates example retention loop structures of an exemplary stent.

FIG. 5B illustrates example retention loop structures of an exemplary stent.

FIG. 5C illustrates another example of a retention loop structure of an exemplary stent.

FIG. 5D illustrates another example of retention loop structures of an exemplary stent.

FIG. 6 illustrates an example stent.

FIG. 7 illustrates a perspective side view of an example distal tip of a stent delivery device including a retaining structure.

FIG. 8 illustrates a perspective side view of an example distal tip of a stent delivery device including retaining structures and a guidewire lumen.

FIG. 9 illustrates a perspective side view of an example distal tip of a stent delivery system including a retaining structure on the distal end, and retaining structures proximal of the distal end.

FIG. 10 illustrates a perspective side view of an example distal tip of a stent delivery device including a retaining structure.

FIG. 11 illustrates a perspective side view of an example distal tip of a stent delivery device including a retaining structure and grooves extending proximally therefrom.

FIG. 12A illustrates a perspective side view of an example distal tip of a stent delivery device including a retention structure, retention barbs, and a guidewire lumen.

FIG. 12B illustrates the distal tip of FIG. 12A with the stent, retention loops, and guidewire in place.

FIG. 13 illustrates a perspective side view of an example distal tip of a stent delivery device including an off-centered retaining structure and guidewire.

FIG. 14A illustrates a perspective side view of an example distal tip of a stent delivery device including a retaining structure, beaked tip and a guidewire lumen.

FIG. 14B illustrates the distal tip of FIG. 14A with a guidewire in place.

FIG. 15 illustrates a perspective side view of an example distal tip of a stent delivery device including retaining structures and a guidewire lumen.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

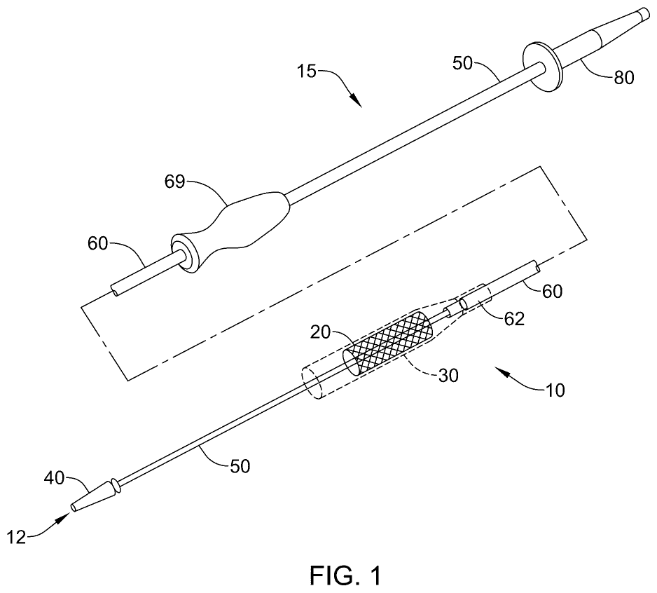

With reference to the drawings, FIG. 1 shows a perspective view of the stent delivery system 10 in accordance with an embodiment of the disclosure. The stent delivery system 10 may include a stent delivery device 15 having a stent 20 loaded therein for delivery to an anatomical site. As seen in FIG. 1, the stent 20 is loaded within an outer sheath 30 which may be attached to or be a portion of an outer tubular member 60 extending proximally to a distal handle 69. In some instances, the outer sheath 30 may be formed separately from the outer tubular member 60 and secured thereto at an attachment joint 62. In other instances, the outer sheath 30 may be a distal end region of the outer tubular member 60 sized to surround the compressed stent 20 prior to deployment. The stent delivery catheter 15 may include an inner member 50 slidably disposed within the lumen of the outer tubular member 60. A distal tip 40 may be secured to or otherwise incorporated with the distal end of the inner member 50. The distal tip 40 may have a distal end 12 at the distalmost extent of the distal tip 40 and the stent delivery device 15. The inner member 50 may extend through the outer tubular member 60 with a proximal end region of the inner member 50 secured to a proximal handle 80. The proximal handle 80 may be located proximal of the distal handle 69 such that the distal handle 69 may be axially translated relative to the proximal handle 80 to longitudinally actuate the outer sheath 60 relative to the stent 20 and the inner member 50, and thus deploy the stent 20. It is noted, that when the stent 20 is radially constrained around the inner member 50 and loaded in the outer sheath 30 for delivery, the distal end of the outer sheath 30 may abut and/or surround a proximal end region of the distal tip 40. While aspects of the present disclosure can be applied to the delivery of many intraluminary devices, it will be described for use in delivering a self-expanding stent 20.

A stent 20 is generally capable of being radially compressed and longitudinally extended when being delivered within the outer sheath 30 for implantation into a body lumen. The degree of elongation may depend upon the structure and intended function of the stent, and may be quite varied. The stent 20 may be constructed to self-expand when released from a radially compressed state (e.g., when a radially restraining force is removed from the stent by withdrawing the outer sheath 30 therefrom). Further, the stent 20 may be repositionable, removeable, and/or reconstrainable in some instances. In some instances, the stent 20 may include one or more, or a plurality of interwoven wires or filaments, forming a braided construction, a knitted construction, or other interwoven construction. In other instances, the stent 20 may be a monolithic structure including a plurality of interconnected struts and interstitial spaces therebetween. Thus, various stent types and constructions may be employed, and the stent delivery system 10 can be constructed to accommodate stents of various sizes and configurations.

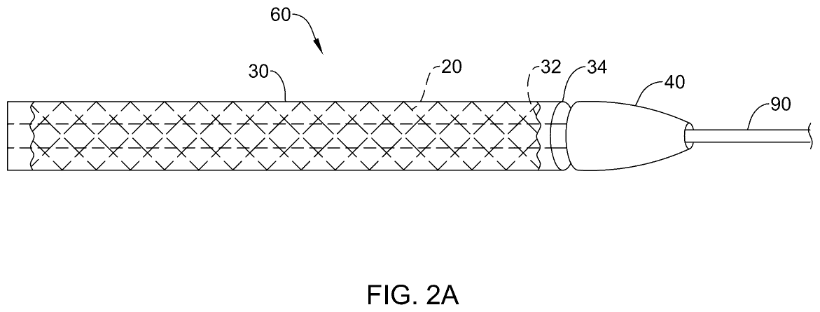

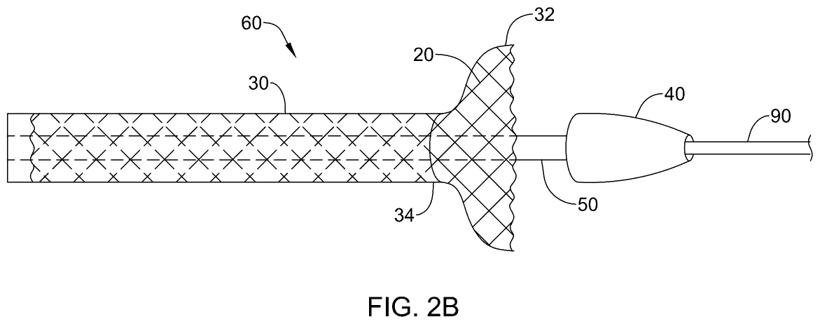

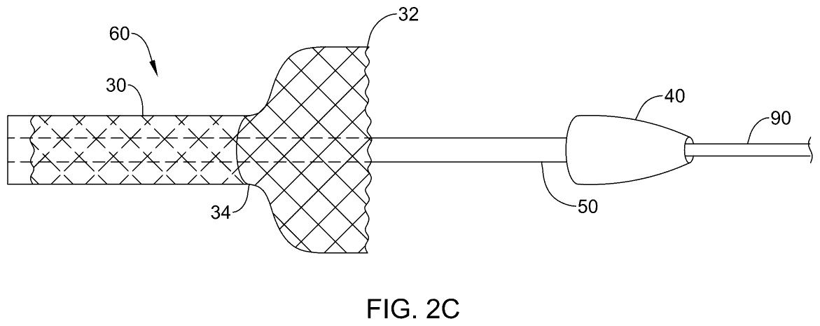

As illustrated in FIGS. 2A-2C, conventional stent delivery systems are prone to stent foreshortening during deployment of the stent. As shown in FIG. 2A, during delivery (e.g., delivery over a guidewire 90), the stent 20 may be positioned in a radially constrained, longitudinally elongated configuration within the distal end region of the outer tubular member 60 (e.g., within the outer sheath 30) such that the radially constrained stent 20 closely surrounds the inner member 50. In the radially constrained configuration, the distal end 32 of the stent 20 is positioned adjacent the distal tip 40. The distal end 34 of the outer sheath 30 may abut and/or surround a proximal end region of the distal tip 40. Withdrawal of the outer tubular member 60 (and thus the outer sheath 30) in the proximal direction relative to the inner member 50, distal tip 40 and the stent 20 moves the distal end 34 of the outer sheath 30 proximal of the distal tip 40 to expose the distal end region of the stent 20. As the outer sheath 30 is proximally withdrawn, the exposed distal end region of the stent 20 is released from the radially constrained configuration, as shown in FIG. 2B. As the exposed distal end region of the stent 20 begins to radially expand, the exposed distal end region of the stent 20 also begins to foreshorten in the proximal direction. In other words, the distal end 32 of the stent 20, when deployed, moves proximally away from the distal tip 40. Further proximal withdrawal of the outer sheath 30, further deploys an additional length of the stent 20 to radially expand as the additional length of the stent 20 is exposed, as shown in FIG. 2C. As the exposed length of the stent 20 increases, the amount of foreshortening of the stent 20 likewise increases, moving the distal end 32 of the stent 20 further proximally away from the distal tip 40 as the stent 20 radially expands and longitudinally foreshortens. In some embodiments, the user may be able to compensate for stent foreshortening by advancing the guidewire 90 and distal tip 40 past the intended deployment location (e.g., stricture) to account for the axial displacement of the distal end of the stent 20 during deployment. However, in many instances, the intended deployment location may be in a small space, or near a location where the distal tip 40 and guidewire 90 are unable to be advanced past the intended deployment location. It can be difficult to access certain areas of the body for stent delivery, due to the foreshortening that occurs upon deployment of the stent 20. Improper stent 20 deployment or placement may cause complications, and/or require removal and replacement of the stent 20.

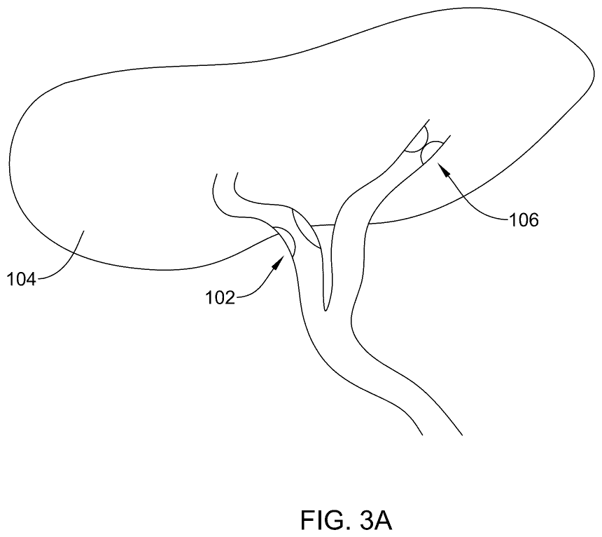

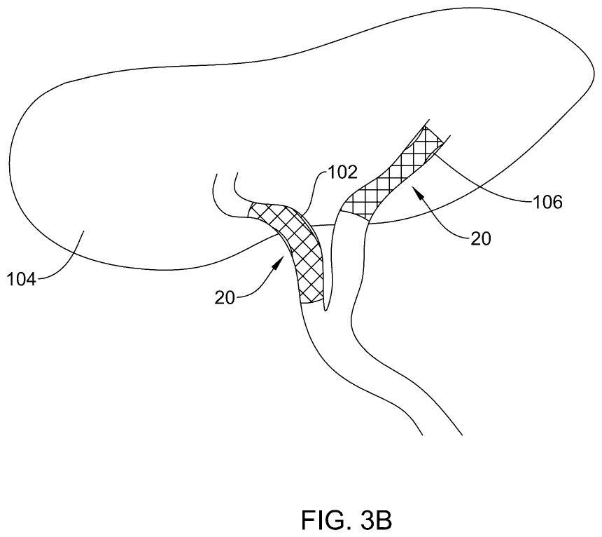

FIG. 3A illustrates an example anatomical location for placing a stent. Strictures 102, 106 are located near an organ 104. As shown in FIG. 3B, the stent 20 placed across the stricture 102 spaced further away from the organ is able to be deployed with the stent 20 approximately centered on the stricture 102. However, the stent 20 placed to treat the stricture 106 located right next to the organ, is unable to be properly centered across the stricture 106, with the majority of the stent 20 extending to one side of the stricture. In such instances, migration of the stent 20 becomes a strong possibility, potentially requiring repositioning and removal of the stent 20. The proximity of the stricture 106 to the organ 104 prevents further advancement of the stent delivery device distal of the stricture 106 to compensate for foreshortening of the stent 20 during stent deployment as discussed above. As described in more detail below, alternative stent delivery devices that prevent or restrict foreshortening (e.g., retain the distal end of the stent 20 closer to the distal tip of the stent delivery device) allow for more accurate stent placement without the recourse of continuous procedural correction or replacement.

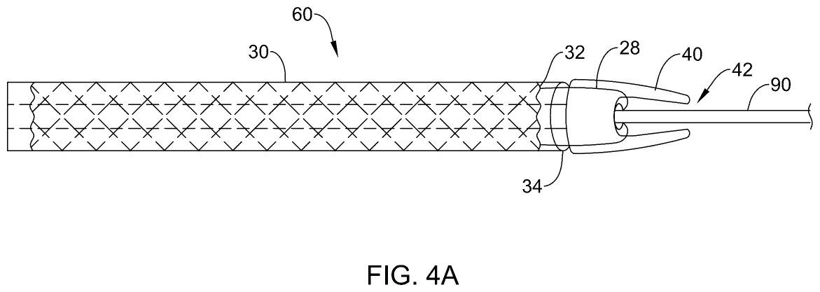

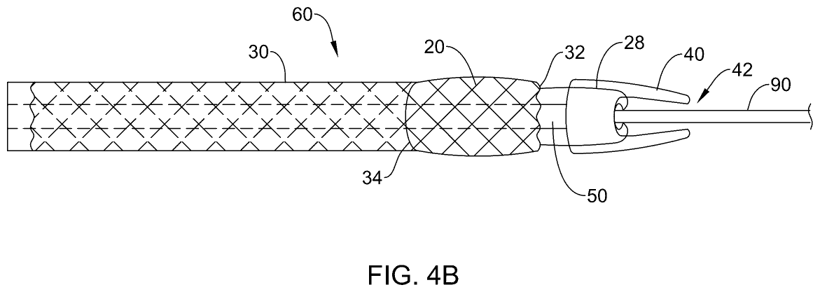

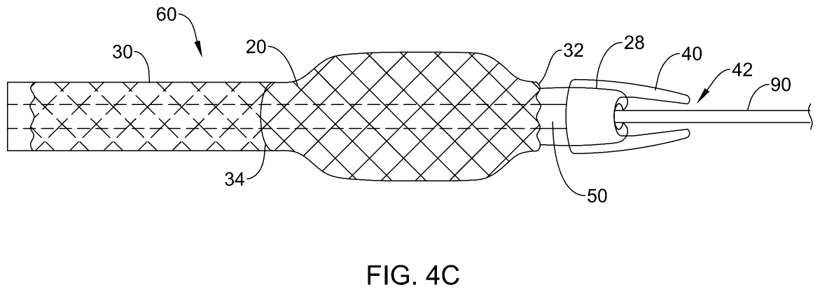

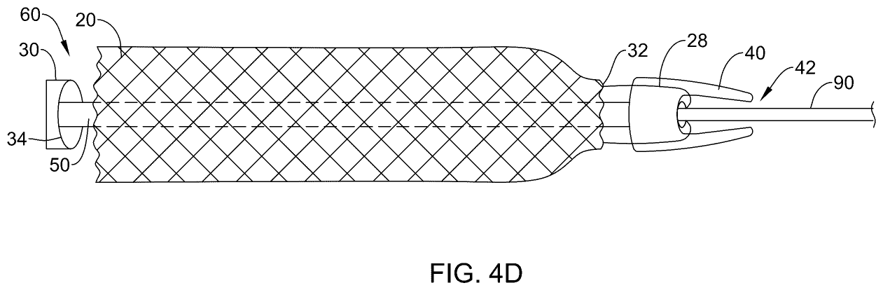

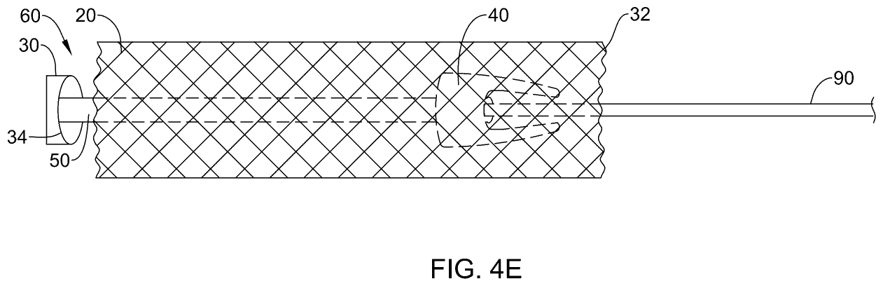

The foreshortening as shown in FIGS. 2A-2C can be remedied with modifications to the distal tip 40 and the stent 20. As shown in FIGS. 4A-4D, the distal tip 40 can be modified to include a retaining structure 42, such as a notch, slit, hook, barb or the like. The stent 20 can be modified to include one or more retaining loops 28, such as sutures, wire loops and the like. The retaining loops 28 can be configured to engage with the retaining structure 42, such that the stent 20 is held in place axially upon proximal retraction of the outer sheath 30/outer tubular member 60, as shown in FIGS. 4B-4D. FIGS. 4B to 4D show the distal end 34 of the outer sheath 30 being withdrawn proximally relative to the stent 20 during deployment of the stent 20. As the distal end of the outer sheath 30 is withdrawn proximally, more of the length of the stent 20 is exposed from the distal end of the outer sheath 30, until the entire length of the stent 20 is exposed, as shown in FIG. 4D. As shown in FIG. 4D, when the entire length of the stent 20 has been exposed by the proximal retraction of the outer sheath 30, the retaining loops 28 of the stent 20 remain engaged with the retaining structure 42 of the distal tip 40 (e.g., the retaining loops 28 remain in the notch formed in the distal tip 40). While the retaining loops 28 are engaged with the retaining structure 42, the distal end 32 of the stent 20 will remain adjacent the distal tip 40, and around an outer surface of the inner tubular member 50, as shown in FIG. 4D throughout deployment of the stent 20. In some embodiments, as the stent 20 is deployed, it radially expands. As the stent 20 radially expands, the distal end 32 of the stent 20 may remain radially constrained by the retaining loops 28 and held adjacent to the distal tip 40.

As show in FIG. 4E, once the entire length of the stent 20 has been exposed form the distal end of the outer sheath 30, and the proximal end of the stent 20 has radially expanded against the body lumen, the inner member 50 and the distal tip 40 may be withdrawn proximally through the lumen of the stent 20. Withdrawing the inner member 50 and the distal tip 40 proximally through he lumen of the stent 20 releases the retaining loops 28 from the retaining structure 42 (e.g., notch) of the distal tip 40, thereby allowing the distal end of the stent 20 to radially expand against the body lumen. Since the notch opens to the distal end of the distal tip 40, the retaining loops 28 may be freed from the retaining structure (e.g., notch) by withdrawing the distal tip 40 proximal of the retaining loops 28 and into the lumen of the stent 20.

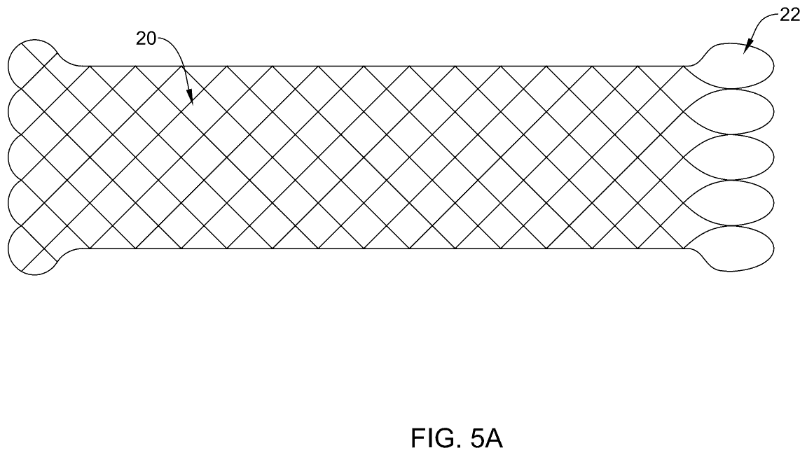

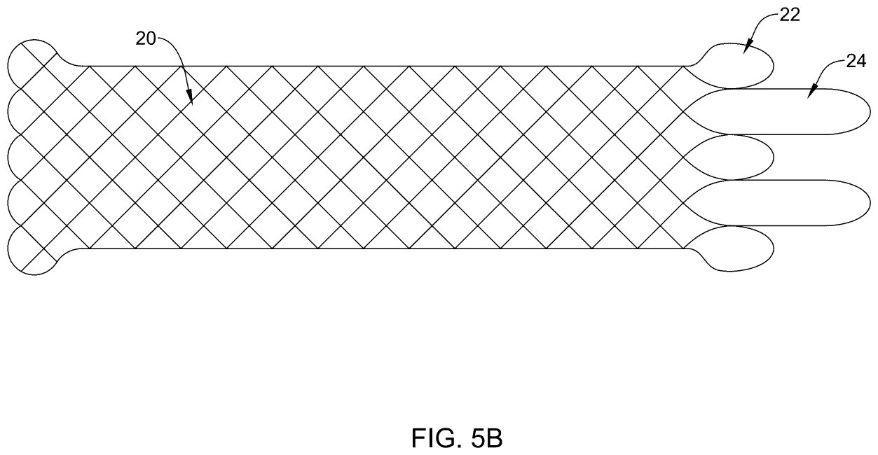

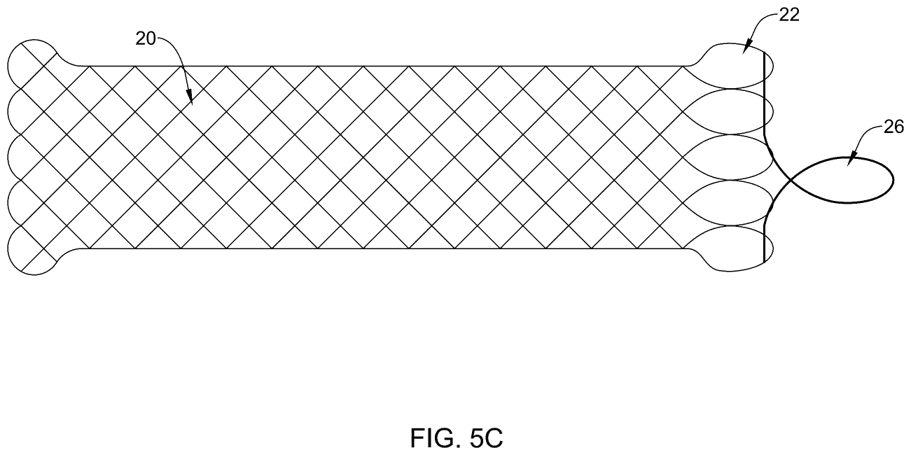

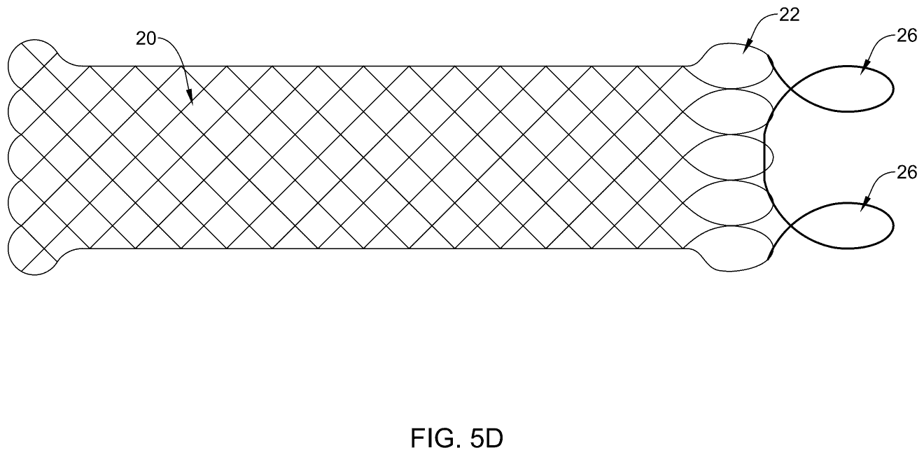

FIGS. 5A-5D illustrate different embodiments of a stent for use in stent delivery systems. The stents 20 include various configurations of retaining loops 22, 24, 26.

In FIG. 5A, the stent 20 includes open loops 22 located at one end of the stent 20. The open loops 22 may be formed of one or more wires used to construct the tubular scaffold of the stent 20. For example, the open loops 22 may be formed of wires extending from the braided, knitted, or otherwise interwoven tubular framework formed of the wires. In some embodiments, the stent 20 may include a coating surrounding at least a portion of the tubular scaffold defining the stent 20. In some instances, the coating on the stent 20 may be removed from portions of the stent 20 defining the open loops 22, or the open loops 22 may otherwise be devoid of the coating. The open loops 22 may be positioned into the retaining structure of the distal tip to retain the stent 20 thereto. In some embodiments, the open loops 22 may be left uncoated. In some embodiments, the size of the loops may be adjusted during the braiding/knitting/weaving process. There may be any number of open loops 22 in any location on the distal end of the stent, and the open loops 22 may be symmetrically or asymmetrically arranged around the circumference of the distal end of the stent 20, as desired.

FIG. 5B illustrates the stent 20 including one or more elongated loops 24 that extend beyond the remainder of the loops 22 at the distal end of the stent 20. The elongated loops 24 may be formed of one or more wires used to construct the tubular scaffold of the stent 20. For example, the elongated loops 24 may be formed of wires extending from the braided, knitted, or otherwise interwoven tubular framework formed of the wires. The size (e.g., length) of the elongated loops 24 may vary to accommodate different retaining structures. In some embodiments, the elongated loops 24 may be formed separately and then added to the tubular framework after construction. The stent 20 may be coated with a coating surrounding at least a portion of the tubular scaffold defining the stent 20. In some instances, the coating on the stent 20 may be removed from portions of the stent defining the elongated loops 24, or the elongated loops 24 may otherwise be devoid of the coating. There may be any number of elongated loops 24, in any location on the distal end of the stent 20. In the embodiments shown in FIGS. 5A-5B, the loops 22, 24 may be formed as part of the stent 20 such that at least some of the wires forming the braided/knitted/interwoven tubular scaffold of the stent 20 also form the loops 22, 24. In other embodiments, the loops 22, 24 may be separate wires fixed (e.g., welded) to the pre-formed tubular scaffold of the stent 20.

FIGS. 5C and 5D illustrate retention loops in the form of loops formed from retention sutures 26, which may be positioned in the retaining structure of the distal tip. The size and position of the suture loops 26 may vary to accommodate the different retaining structures. There may be any number of sutures 26, in any location on the distal end of the stent 20. The suture forming the suture loops 26 may extend around the circumference of the distal end of the stent 20, such as being interwoven through one or more loops in the tubular scaffold of the stent 20. In some instances, the sutures 26 may be woven around the circumferential edge of the stent 20 at the distal end thereof. A portion of the suture 26 may extend from the circumferential edge and define a loop of the suture 26 configured to be positioned in the retaining structure of the distal tip of the delivery device. In some embodiments, the sutures 26 may be woven through the length of the stent 20. As shown in FIG. 5C, in some instances, the suture 26 may form only a single suture loop extending from the tubular scaffold of the stent 20, whereas FIG. 5D illustrates a configuration in which the suture 26 forms a plurality of suture loops extending from the tubular scaffold of the stent 20. Multiple suture loops may be symmetrically or asymmetrically arranged around the circumference of the distal end of the stent 20.

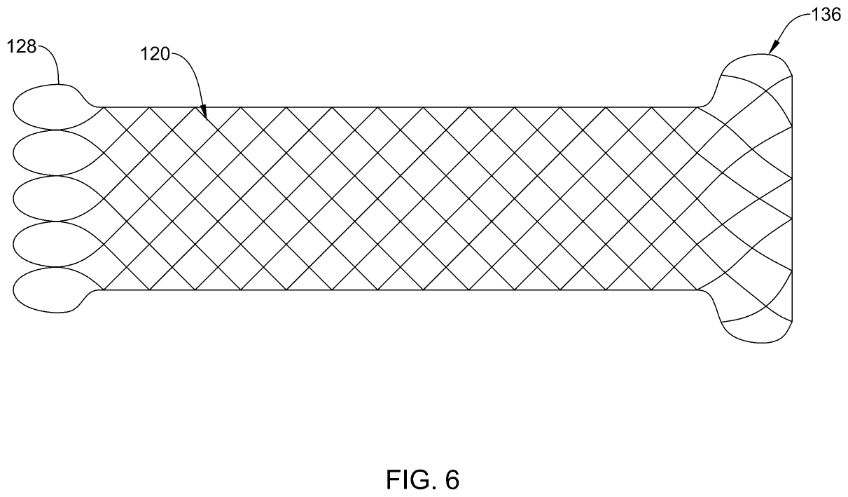

FIG. 6 illustrates another configuration of a stent 120 which may be used, for example as a hepaticogastrostomy (HGS) stent. The flared end 136 of the HGS stent 120 may be configured to prevent migration of the stent once it has been placed in a body lumen. The stent 120 may include retaining loops 128. In some instances, the loops 128 may be on an end of the stent that is not flared. In some embodiments the loops 128 may be on the end of the stent that is flared 136. The retaining loops 128 may be formed of one or more wires forming the tubular scaffold of the stent 120 and extending therefrom, or the retaining loops 128 may be formed of a filament, e.g., a suture or wire, interconnected to the end region of the stent 120. For instance, the retaining loops 128 may be any of the open loops, elongated loops, and sutures as described above. There may be any number of retaining loops 128 at any location along the stent 120. The retaining loops 128 may be configured to be positioned in the retaining structure of the distal tip of the stent delivery device, as will be discussed in more detail below.

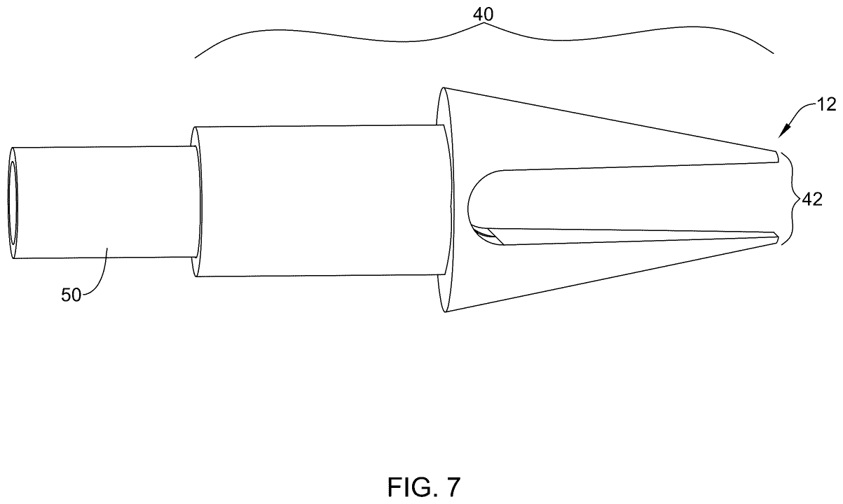

FIG. 7 illustrates an example distal tip 40 of the stent delivery system 10. The distal tip 40 may be disposed at the distal end of the inner member 50. In some embodiments the distal tip 40 and inner member 50 are two separate pieces that have been connected together. For example, the distal tip 40 may be separately formed and secured to the distal end of the inner member 50. In some embodiments the distal tip 40 and the inner member 50 are a single monolithic element. The distal tip 40 may include a retaining structure 42. The retaining structure may comprise a notch 42, extending proximally from the distal end 12 of the distal tip 40, with an access opening of the notch 42 located at the distal end 12 of the distal tip 40. The opening of the notch 42 may extend across the width of the distal end of the distal tip in a direction generally transverse to the longitudinal axis of the inner member 50. The notch 42 may extend proximally and be of a depth sufficient to hold in place one or more retention loops of the stent 20, as discussed above. In some embodiments, the notch 42 may be of any depth, such as a depth greater than one-half the width (e.g., at least one-half the diameter) of the retention loop. In some embodiments, the notch 42 may extend one-half or more of the length of the distal tip 40. The notch 42 may be wide enough to accommodate any number of retention loops, and/or a guidewire. The retention loops of the stent may be inserted into the notch 42 and/or removed from the notch 42 from the distal opening of the notch 42 at the distal end 12 of the distal tip 40, with the retention loops extending from the opposing sides of the notch 42.

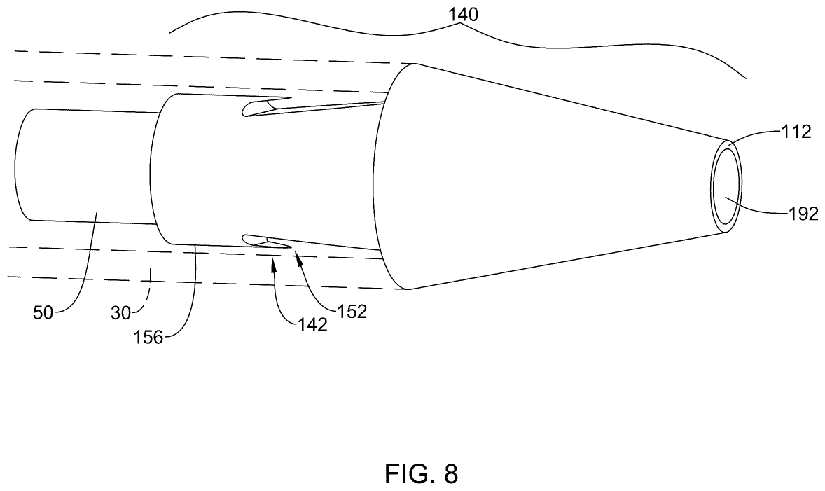

FIG. 8 illustrates another example distal tip 140 of the stent delivery system 10. The distal tip 140 may be disposed at the distal end of the inner member 50. In some embodiments the distal tip 140 and inner member 50 are two separate pieces that have been connected together. For example, the distal tip 140 may be separately formed and secured to the distal end of the inner member 50. The distal tip 140 may include a guidewire lumen 192 extending therethrough configured to accommodate a guidewire extending through the lumen of the inner member 50 and distal of the distal tip 140. In some embodiments the distal tip 140 and the inner member 50 are a single monolithic element. The distal tip 140 may include a retaining structure 142. The retaining structure 142 may comprise one or more notches 152 disposed on a portion of the distal tip 140 proximal of the distal end 112 of the distal tip 140. For example, the one or more notches 152 may extend into a sidewall of the distal tip from an outer surface 156 of the distal tip 140. The one or more notches 152 may extend proximally from their starting point at the outer surface 156 at an angle relative to the longitudinal axis of the inner member 50 and/or the distal tip 140. Thus, the notches 152 may extend radially inward from the outer surface 156 of the distal tip 140. The one or more notches 152 could also include a hook, barb, or ridge extending radially outward from the outer surface 156 of the distal tip 140. The notches 152 may be of a depth sufficient to hold in place one or more retention loops of the stent. In some embodiments, the notch 152 may be of any depth, such as a depth greater than one-half the width (e.g., at least one-half the diameter of the retention loop. The size of the notch 152 may vary depending upon the stent and delivery location. The notch 152 may be shallow enough, so that when the stent has been deployed, the retaining loops will decouple or otherwise disengage from the notch 152 (e.g., slide out of the notch 152) upon proximal retraction of the inner member 50 and distal tip 40 relative to the deployed, radially expanded stent.

In some instances, the outer sheath 30 (shown in dashed lines) may extend over the outer surface 156 having the notches 152 formed therein when the stent is loaded within the outer sheath 30 during delivery of the stent. The placement of the outer sheath 30 around the outer surface 156 of the distal tip 140 may help retain the retaining loops of the stent within the notches 152 until the outer sheath 30 is withdrawn proximally to uncover the notches 152 and the stent.

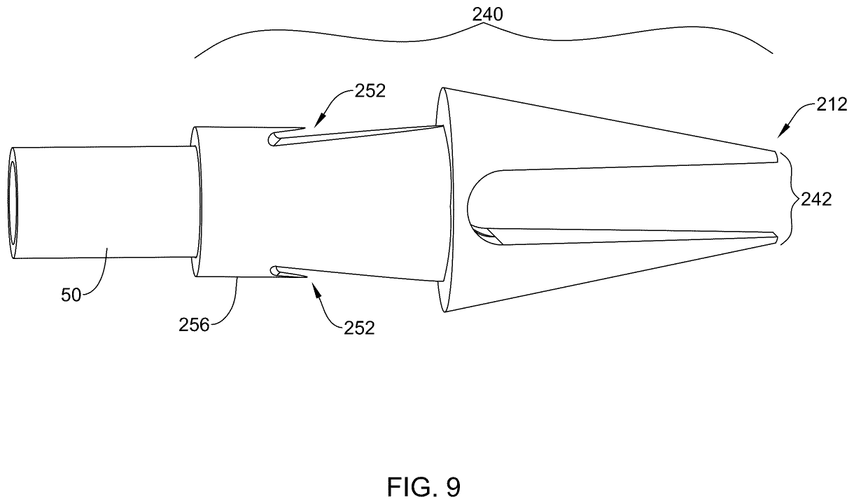

FIG. 9 illustrates an example distal tip 240 of the stent delivery system 10. The distal tip 240 may be disposed at the distal end of the inner member 50. In some embodiments the distal tip 240 and inner member 50 are two separate pieces that have been connected together. For example, the distal tip 240 may be separately formed and secured to the distal end of the inner member 50. In some embodiments the distal tip 240 and the inner member 50 are a single monolithic element. The distal tip 240 may include a retaining structure, in the form of a notch 242, similar to that described in association with FIG. 7. The notch 242 may open out to the distal end 212 of the distal tip 240. In some embodiments, the retaining structure may further include the notches 252 formed in the outer surface 256 of the distal tip 240 proximal of the distal end 212 of the distal tip 240. The notches 252 may be hooks, barbs, ridges, and/or grooves as described in FIG. 8. The notches 242, 252 may be of a depth sufficient to hold in place one or more retention loops of the stent. The size of the notches 242, 252 may vary depending upon the stent and delivery location. The notch may be shallow enough, so that when the stent has been deployed and radially expanded in a body lumen, the retaining loops of the stent will slide out of the notches upon proximal retraction of the inner member 50 and distal tip 240 relative to the radially expanded, deployed stent.

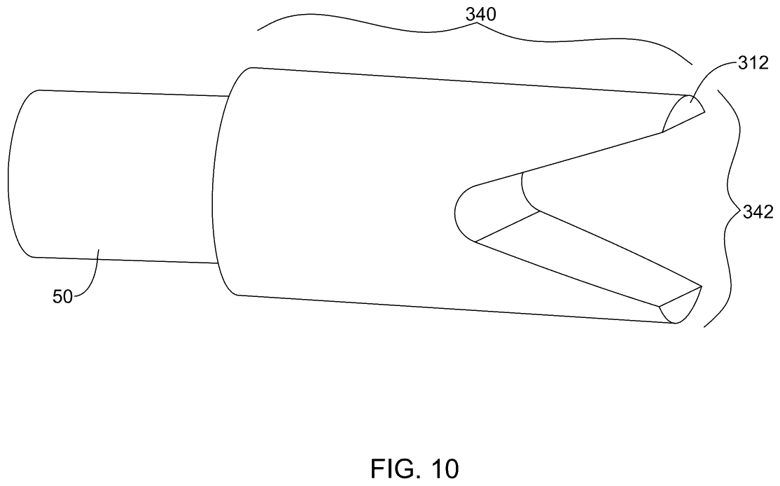

FIG. 10 illustrates another example distal tip 340 of the stent delivery system 10. The distal tip 340 may be disposed at the distal end of the inner member 50. In some embodiments the distal tip 340 and inner member 50 are two separate pieces that have been connected together. For example, the distal tip 340 may be separately formed and secured to the distal end of the inner member 50. In some embodiments the distal tip 340 and the inner member 50 are a single monolithic element. The distal tip 340 may include a retaining structure. For example, the retaining structure may comprise one or more notches 342 extending proximally from the distal end 312 of the distal tip 340. The notch 342 may begin at the distal end 312 of the distal tip and extend proximally to a base of the notch 342. The notch may taper proximally, such that the opening of the notch 342 at the distal end 312 of the distal tip 342 is wider than the base of the notch 342. For example, the side surfaces defining the notch 342 may extend at an acute angle to the central longitudinal axis of the distal tip 340. The notch 342 may have a width at the distal end 312, measured transverse to the longitudinal axis, that is greater than the width of the notch 342 at the proximal end of the notch. In some instances, the notch 342 may be V-shaped. The notch 342 may be of a depth sufficient to hold in place one or more retention loops with the retention loop(s) extending laterally from opposite sides of the notch 342. In some embodiments, the notch 342 may be of any depth, such as a depth greater than one-half the width of the retention loop. The size of the notch 342 may vary depending upon the stent and delivery location. The notch 342 may be shallow enough, so that when the stent has been deployed and radially expanded in a body lumen, the retaining loops of the stent will slide out of the notch 342 upon proximal retraction of the inner member 50 and distal tip 340 relative to the radially expanded, deployed stent.

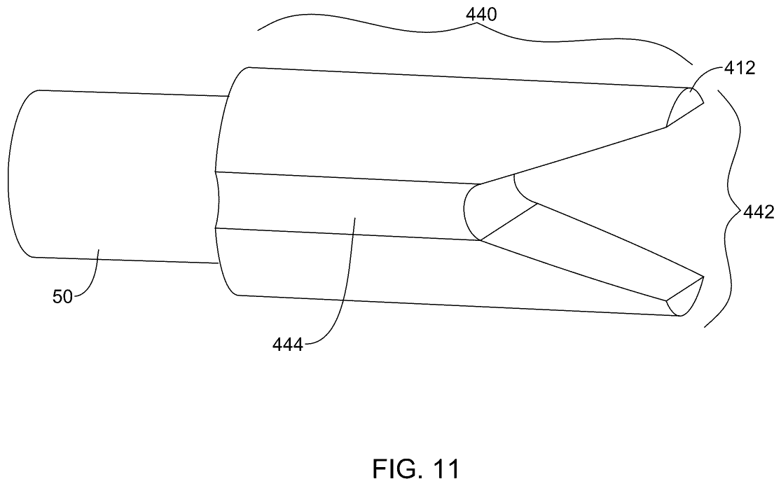

FIG. 11 illustrates another distal tip 440 that may be disposed at the distal end of the inner member 50. The distal tip 440 may be similar to that shown in FIG. 10, including a notch 442 extending proximally from a distal end 412 of the distal tip 440 to a base of the notch 442, with the addition of a longitudinal groove 444 along the outer surface of the distal tip 440 extending proximally from the base of the notch 442. Although not shown in FIG. 11, the distal tip 440 may include a second longitudinal groove 444 extending along the outer surface of the distal tip 440 on the opposite side of the distal tip 440. For example, the distal tip 440 may include first and second longitudinal grooves 444 on opposing sides of the distal tip 440, with both grooves 444 extending proximally from the base of the notch 442. In some instances, the first and second longitudinal grooves 444 may be diametrically opposite one another. The grooves 444 may extend parallel to a longitudinal axis of the distal tip 440. The grooves 444 may be sized and shaped to accommodate portions of the retention loops extending from opposite sides of the notch 442 as the retention loops extend along the distal tip proximal of the notch 442. The grooves 444 may be placed in a location such that when the retention loops are engaged with the retention structure, the retention loops rest within the grooves 444. In some instances, the grooves 444 may be a depth such that when one or more retention loops are resting within the grooves 444, the retention loops do not extend radially past the outer diameter of the distal tip 440.

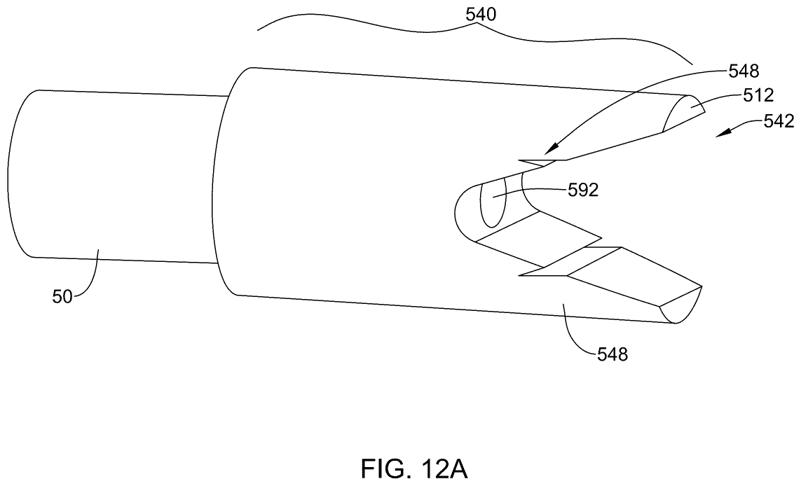

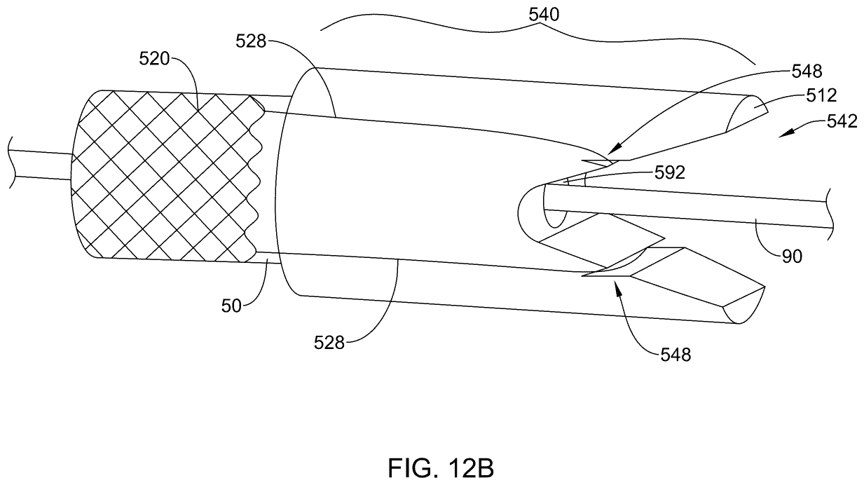

FIG. 12A illustrates another example distal tip 540, similar to the distal tip of FIG. 10 including a retaining structure in the form of a notch 542, with an added guidewire lumen 592 extending through the distal tip 540 with a distal opening at the base of the notch 542, located at the proximal end of the notch 542. The guidewire lumen 592 may be sized and shaped to accommodate any guidewire passing through the lumen of the inner member 50 and the lumen 592. The distal tip 540 may further include retention barbs 548 located along the notch 542. The barbs 548 may be sized and shaped to hold the retention loops of the stent in place. There may be any number of barbs 548 located along the notch 542. For example, each of the side surfaces defining the notch 542 may include a barb 548. The barb 548 may define a groove sized to receive a retention loop of the stent. As shown in more detail in FIG. 12B, when the retention loops 528 of the stent 520 are engaged with the retention structure of the distal tip 540, the retention loops 528 may be held in place by the retention barbs 548. For example, the retention loops 528 may be passed through the grooves defined by the barbs 548. The guidewire 90 may extend from the distal opening of the guidewire lumen 592, through the notch 542, and distally from the distal end 512 of the distal tip 540. In some instances, it may be advantageous to avoid the retention loops 528 coming in contact with the guidewire 90. Accordingly, placement of the retention loops 528 in the grooves formed by the barbs 548 may avoid the retention loops 528 getting entangled with, impeding movement of, or otherwise contacting the guidewire 90. The retention barbs 548 may be integrated in any of the distal tip retaining structures as described herein.

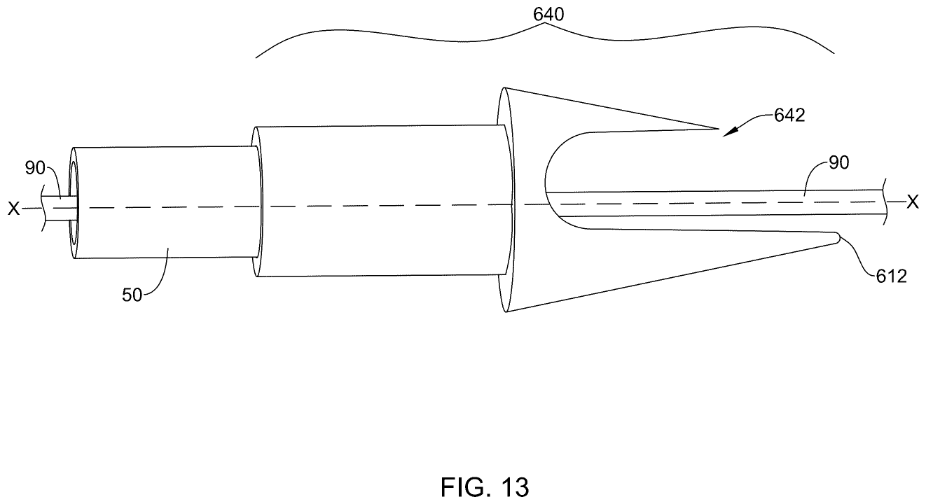

FIG. 13 illustrates another example distal tip 640 that may be disposed at the distal end of the inner member 50 of the stent delivery system 10. In some embodiments the distal tip 640 and inner member 50 are two separate pieces that have been connected together. For example, the distal tip 640 may be separately formed and secured to the distal end of the inner member 50. In some embodiments the distal tip 640 and the inner member 50 are a single monolithic element. The distal tip 640 may include a retaining structure. For example, the retaining structure may comprise a notch 642, extending proximally from the distal end 612 of the distal tip 640 and extend proximally to a base of the notch 642. The distal tip 640 may also include a guidewire lumen, which may be similar to the guidewire lumen 592 described above in association with FIGS. 12A-12B, configured to accommodate a guidewire 90 extending therethrough. The guidewire lumen, and thus the guidewire 90, may extend along a central longitudinal axis X-X, which the notch 642 may be off-centered from the central longitudinal axis X-X. In other words, the notch 642 may be shifted laterally away from the central longitudinal axis X-X such that the guidewire extends along one sidewall of the notch 642 and is space further from the opposing sidewall of the notch 642. The notch 642 may be of a depth sufficient to hold in place one or more retention loops of a stent. In some embodiments, the notch 642 may be of any depth, such as a depth greater than one-half the width of the retention loop. The notch 642 may be wide enough to accommodate any number of retention loops, and/or a guidewire. The notch 642 may be sufficiently off centered from the central longitudinal axis X-X such that when the guidewire 90 extends through the notch 642, the retention loops are spaced away from and will not come in contact with the guidewire 90. The guidewire 90 may be located on one side of the notch 642, with the other side of the notch 642 being empty to accommodate the retention loops of the stent.

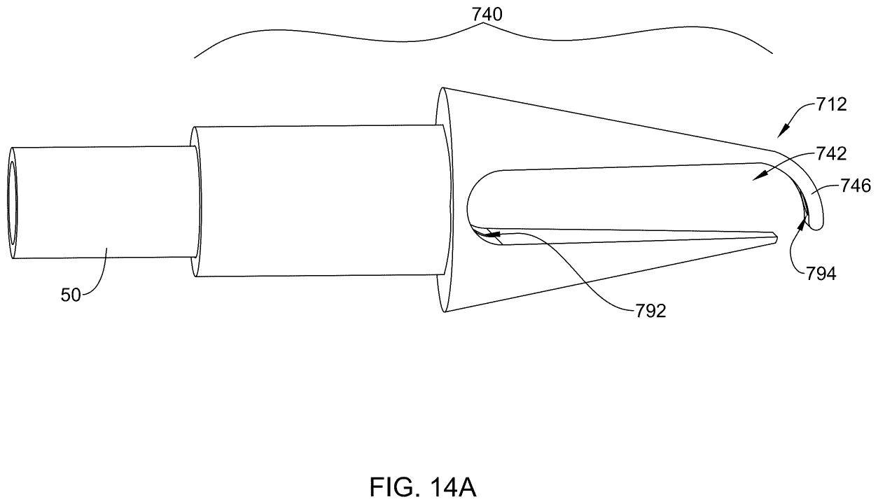

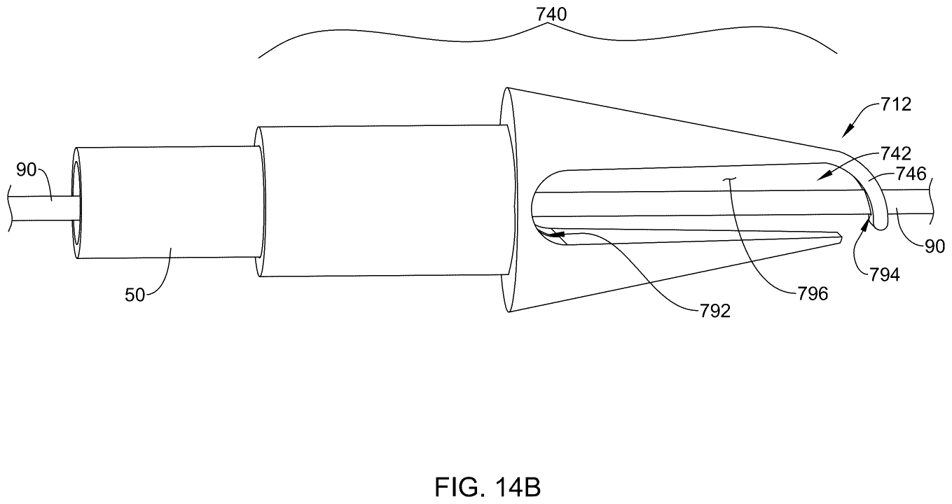

FIG. 14A illustrates another example distal tip 740 of the stent delivery system 10. The distal tip 740 may be disposed at the distal end of the inner member 50. In some embodiments the distal tip 740 and inner member 50 are two separate pieces that have been connected together. For example, the distal tip 740 may be separately formed and secured to the distal end of the inner member 50. In some embodiments the distal tip 740 and the inner member 50 are a single monolithic element. The distal tip 740 may include a retaining structure for retaining one or more retaining loops of the stent. The retaining structure may comprise a notch 742, extending proximally from the distal end 712 of the distal tip 740. In some instances the notch 742 may be centered along a central longitudinal axis of the distal tip 740. The distal tip 740 may also include a guidewire lumen 792, which may be similar to the guidewire lumens described above, configured to accommodate a guidewire 90 extending therethrough. The guidewire lumen 792, and thus the guidewire 90, may extend along a central longitudinal axis of the inner member 50 and/or the distal tip 740. The distal tip 740 may further include a curved beak 746. The curved beak 746 may extend to the distal end of the notch 742, and be curved to cover or extend laterally across at least a portion of the distal opening of the notch 742. In some instances, the curved beak 746 may further include a guidewire lumen 794 through which a guidewire 90 may extend, as shown in FIG. 14B. When the guidewire 90 is extending through the lumen, it may partially block the opening of the notch 742.

When the retention loops of the stent are positioned in the notch 742 and the distal tip 740 is tracked along a guidewire 90 during delivery of the stent with the guidewire extending through the guidewire lumen 792 of the distal tip 740 and the guidewire lumen 794 of the curved beak 746, the retention loops may be positioned in the bounded opening 796 of the notch 742. The bounded opening 796 may be defined between the base of the notch 742 and the curved beak 742, and between the guidewire 90 and a sidewall of the notch 742. Such a configuration may be advantageous to prevent the retention loops from coming out of the notch 742 before intended. For example, the positioning of the guidewire 90 through the guidewire lumen 794 of the curved beak 746 may prevent the retention loops from coming out of the notch 742 until the guidewire 90 has been withdrawn proximally and removed from the guidewire lumen 794 of the beak 746. The curved beak 746 may be formed of a flexible material, such as a flexible polymer material, such that the curved beak 746 may flex or be deflected to allow the retention loops of the stent to be released from the notch 742 when the guidewire 90 is withdrawn and the distal tip 740 is moved proximally relative to the stent during deployment of the stent.

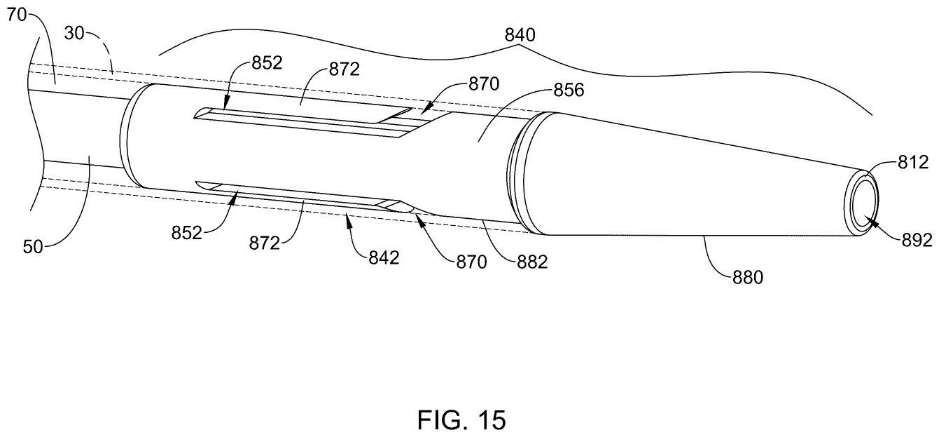

FIG. 15 illustrates another example distal tip 840 of the stent delivery system 10. The distal tip 840 may be disposed at the distal end of the inner member 50. In some embodiments the distal tip 840 and inner member 50 are two separate pieces that have been connected together. For example, the distal tip 840 may be separately formed and secured to the distal end of the inner member 50. In some embodiments the distal tip 840 and the inner member 50 are a single monolithic element.

The distal tip 840 may include a frustoconically tapered distal region 880 extending to the distal end 812 of the distal tip 840. The tapered distal region 800 may taper distally to a smaller diameter to facilitate advancing the stent delivery system 10 through a body lumen. The distal tip 840 may include a cylindrical proximal region 882 extending proximal from the tapered distal region 880. The proximal region 882 may include a circumferential outer surface 856. In some instances, the diameter of the proximal region 882 may be less than the greatest diameter of the tapered dial region 880, such as at its proximalmost extent. The distal tip 840 may include a guidewire lumen 892 extending therethrough and in communication with the lumen of the inner member 50. The guidewire lumen 892 may extend to a distal open at the distal end 812 of the distal tip 840. The guidewire lumen 892 may be sized to accommodate a guidewire extending therethrough.

The distal tip 840 may include a retaining structure 842. The retaining structure 842 may comprise one or more notches 852 disposed on a portion of the distal tip 840 proximal of the distal end 812 of the distal tip 840. For example, the one or more notches 852 may extend into a sidewall of the distal tip from an outer surface 856 of the distal tip 840 at an opening 870 at a distalmost extent of the notches 852. The one or more notches 852 may extend proximally from their starting point (i.e., the opening 870) at the outer surface 856 at an angle relative to the longitudinal axis of the inner member 50 and/or the distal tip 840. Thus the notches 852 may extend inward from the outer surface 856 of the distal tip 840 at an oblique angle to the central longitudinal axis of the distal tip 840. The notches 852 may also include a longitudinal portion extending proximally from the angled distal portion of the notches 852. The proximal longitudinal portion of the notches may extend generally parallel to the central longitudinal axis of the distal tip 840 in some instances. The longitudinal portion of the notches 852 may extend proximally to a base of the notches 852. Accordingly, the base of the notches 852 may be located proximal of the opening 870 of the notches 852, with the base of the notch 852 and the openings 870 located at opposite ends of the notch 852. The inclusion of the notches 852 may form tabs 872 from a portion of the tubular wall of the proximal region 882 of the distal tip 840. The base of the tabs 872 where the tabs 872 are joined to the remainder of he proximal region 882 may be located at the base of the notches 852, while the tips of the tabs 872 may be located at the opening 870.

The notches 852 may be of a width and/or length sufficient to hold in place one or more retention loops of the stent. In some embodiments, the notch 852 may be of any width and/or length, such as a width and/or length greater than one-half the width (e.g., at least one-half the diameter of the retention loop. The size of the notch 852 may vary depending upon the stent and delivery location. The notch 852 may be shallow enough, so that when the stent has been deployed, the retaining loops will decouple or otherwise disengage from the notch 852 (e.g., slide out of the notch 852) through the opening 870 upon proximal retraction of the inner member 50 and distal tip 40 relative to the deployed, radially expanded stent.

The delivery system may define a stent receiving region 70 defined between an inner surface of the outer sheath (shown in dashed lines) and an outer surface of the inner member 50. The stent receiving region 70 may be located proximal of the distal tip 840. A stent (not shown) may be positioned in the stent receiving region 70 in a radially constrained configuration with one or more retention loops extending distally therefrom and engaged in the notches 852 of the distal tip 840. In some instances, the outer sheath 30 may extend over the outer surface 856 having the notches 852 formed therein when the stent is loaded within the outer sheath 30 (i.e., loaded in the stent receiving region 70) during delivery of the stent. For example, the outer sheath 30 may extend over the proximal region 882 with a distal end of the outer sheath 30 abutting a proximal shoulder of the tapered distal region 880 of the distal tip 840. The placement of the outer sheath 30 around the outer surface 856 of the distal tip 840 may help retain the retaining loops of the stent within the notches 852 until the outer sheath 30 is withdrawn proximally to uncover the notches 852 and the stent. For example, placement of the outer sheath 30 over the tabs 872 and across the opening 870 of the notches 852 may capture the retaining loops of the stent within the notches 852 until the proximal end of the stent is fully exposed and radially deployed. At that point, the inner member 50 and distal tip 840 may be withdrawn proximally through the lumen of the deployed stent such that the retaining loops of the stent 852 move distally through the longitudinal portion of the notches 852 and exit out the openings 870 of the notches 852 to release the retaining loops from the distal tip 840 and fully decouple the stent from the stent delivery device.

It will be understood that the dimensions described in association with the above figure are illustrative only, and that other dimensions of slits and filter sheaths are contemplated. The materials that can be used for the various components of the stent delivery device for capturing lesion particles (and/or other systems or components disclosed herein) and the various elements thereof disclosed herein may include those commonly associated with medical devices. For simplicity purposes, the following discussion makes reference to the stent delivery device (and variations, systems or components disclosed herein). However, this is not intended to limit the devices and methods described herein, as the discussion may be applied to other elements, members, components, or devices disclosed herein.

In some embodiments, the stent delivery device (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the stent delivery device (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent delivery device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery device (and variations, systems or components thereof disclosed herein) to achieve the same result.

In some embodiments, the stent delivery device (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the stent delivery device (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent delivery system, the system comprising:
an outer tubular member having a proximal end, a distal end, and a lumen extending to the distal end;
an inner member slidably disposed within the lumen of the outer tubular member;
a distal tip disposed at a distal end of the inner member, the distal tip including a retaining structure; and
a self-expanding stent having a proximal end, a distal end, and a lumen extending therethrough, the stent being expandable from a radially compressed configuration to a radially expanded configuration, the stent including one or more retaining loops disposed at the distal end of the stent;
wherein in the radially compressed configuration, the self-expanding stent is disposed between an outer surface of the inner member and an inner surface of the outer tubular member;
wherein the one or more retaining loops of the stent is configured engage with the retaining structure of the distal tip to maintain position of the distal end of the stent relative to the distal tip.

2. The system of claim 1, wherein the retaining structure comprises a notch opening out to a distal end of the distal tip.

3. The system of claim 1, wherein the retaining structure comprises at least one notch opening out to a side surface of the distal tip proximal a distal end of the distal tip.

4. The system of claim 1, wherein the distal tip further comprises a longitudinal groove extending along a side surface of the distal tip, wherein the one or more retention loops is configured to rest within the groove.

5. The system of claim 1, wherein the one or more retaining loops of the self-expanding stent comprise at least one elongated loop.

6. The system of claim 1, wherein the one or more retaining loops of the self-expanding stent comprise at least one retaining suture.

7. The system of claim 1, further comprising a guidewire slidably disposed within a lumen of the inner member.

8. The system of claim 7, wherein the distal tip of the inner member further comprises a guidewire lumen through which the guidewire can extend in a distal direction past the distal end of the distal tip.

9. The system of claim 8, wherein the notch is off-centered relative to a central longitudinal axis of the distal tip, such that when the guidewire moves in a proximal or distal direction, the guidewire does not come in contact with the one or more retaining loops disposed within the notch.

10. The system of claim 2, wherein the distal tip further comprises a curved beak configured to partially cover the notch.

11. The system of claim 10 wherein the curved beak of the distal tip comprises a guidewire lumen.

12. A stent delivery system, the system comprising:
an outer tubular member having a proximal end, a distal end, and a lumen extending to the distal end;
an inner member slidably disposed within the lumen of the outer tubular member;
a distal tip disposed at a distal end of the inner member, the distal tip including a notch;
a self-expanding stent having a proximal end, a distal end, and a lumen extending therethrough, the stent being expandable from a radially compressed configuration to a radially expanded configuration, the stent including one or more retaining loops disposed at the distal end of the stent;
wherein in the radially compressed configuration, the self-expanding stent is disposed between an outer surface of the inner member and an inner surface of the outer tubular member;
wherein the one or more retaining loops of the self-expanding stent are positionable in the notch of the distal tip;
wherein the outer tubular member is configured to be withdrawn proximally relative to the stent to expose the stent for radial expansion to the radially expanded configuration;
wherein the one or more retaining loops is disposed within the notch to hold the distal end of the stent in place axially relative to the distal tip when the outer tubular member is withdrawn proximally.

13. The system of claim 12, wherein the notch opens out in a distal direction.

14. The system of claim 13, wherein the one or more retaining loops of the self-expanding stent are releasable from the notch when the distal tip is withdrawn proximally through the lumen of the stent.

15. The system of claim 12, wherein the distal tip further comprises a curved beak configured to partially cover the notch.

16. The system of claim 15, wherein the curved beak of the distal tip comprises a guidewire lumen configured to receive a guidewire therethrough.

17. A method of deploying a stent, the method comprising:
advancing a distal end of a stent delivery device to a target location within a body lumen, the stent delivery device including an outer tubular member, an inner member slidably disposed within the outer tubular member, and a distal tip disposed at a distal end of the inner member, a stent loaded within the outer tubular member proximal of the distal tip, the stent including one or more retaining loops engaged with a retention feature of the distal tip of the stent delivery device;
withdrawing the outer tubular member of the stent delivery device in a proximal direction relative to the stent;
wherein withdrawal of the outer tubular member from the stent exposes an entire length of the stent to permit a proximal end region of the stent to radially expand to an expanded configuration while the one or more retaining loops remain engaged with the retention feature of the distal tip;
thereafter, withdrawing the inner member in the proximal direction to release the one or more retaining loops of the stent from the retaining structure of the distal tip.

18. The method of claim 17, wherein the retaining structure comprises a notch opening out in a distal direction.

19. The method of claim 18, wherein upon withdrawing the inner member, the self-expanding stent is held frictionally in place within the body, such that the one or more retaining loops are released from the notch.

20. The method of claim 17, wherein upon releasing the one or more retaining loops from the retaining structure, a distal end of the stent is allowed to radially expand against the body lumen.
